(19) United States
(12) Patent Application Publication
(10) Pub. No.: US 2022/0211122 A1
(43) Pub. Date: Jul. 7, 2022

(54) Garment

(71) Applicant: Alignmed, Inc., Santa Ana, CA (US)

(72) Inventor: William J. Schultz, Newport Beach, CA (US)

(21) Appl. No.: 17/454,802

(22) Filed: Nov. 12, 2021

Related U.S. Application Data
(63) Continuation of application No. PCT/US20/32753, filed on May 13, 2020.
(60) Provisional application No. 62/847,210, filed on May 13, 2019.

(51) Int. Cl. A41D 13/00

(52) U.S. Cl. CPC A41D 13/0002; A41D 2400/32

(57) ABSTRACT

A garment is provided for selective attachment of support straps. The straps may be integrated into the garment or may separate therefrom. The straps may be used for support of the wearer. The straps may be used to customize and/or fit the garment to the wearer.

### PRIORITY

The instant application is a continuation of International Application Number PCT/US20/32753 filed May 13, 2020, published as WO 2020/232172 which claims priority to U.S. Provisional Patent Application No. 62/847,210, filed May 13, 2019, each of which are incorporated by their entirety herein.

### BACKGROUND

In general garments are worn by persons for different purposes including protection from environmental elements, modesty, adornment and fashion. Some garments are worn to support, or to influence neuromuscular activity for therapeutic results, or to improve physical appeal system. An example is a form-fit garment, worn to aid muscle balance, body alignment, circulation and/or postural fitness. A form-fit garment may be used as an adjunctive treatment for chronic pain, injury, disease and disorder.

Taping techniques have been used for many generations to provide an exterior support and stability to muscles and joints to assist and facilitate the body's natural healing processes. Techniques similar to taping have been applied to exterior garments worn by a user. These garments provide a close fitting apparel that either have bands or restrictive mechanisms integrated therein and/or in which separate bands are applied thereto. The apparel provides a base to attach or secure the bands so that contact or adhesion to the skin is not necessary. However, the idea is the same, the bands are configured to applied support and/or pressure to the body by attaching bands at different points on the body.

When using apparel as an intermediary to taping or applying pressure to or pull on body parts, the obvious drawback is the movement of the apparel relative to the body. Another common issue is the selection or customization of the garment to the individual body style. Body styles of individuals vary drastically and are generally as unique as the person themselves. In addition, the purpose and need of the individual is just as unique. For example, two people may desire a garment to improve posture, but the body styles between the two may be very different or the alignment that is desired to improve the posture may be very different. The difference can still be present even if the two people have the same measurements or garment size.

### BRIEF SUMMARY

Exemplary embodiments described herein include different design shapes for garments.

Exemplary embodiments relate generally to a garment or device worn by a person for functional improvements in postural form, health and fitness, comfort, range of motion, reduced interference with range of motion, and combinations thereof.

### BRIEF DESCRIPTION OF THE DRAWINGS

FIG. 1 illustrates an exemplary garment according to embodiments described herein including a support band having a plurality of attachment points.

FIG. 1A illustrates a close up of the exemplary garment of FIG. 1.

FIG. 1B illustrates the exemplary garment of FIG. 1 with an insert according to embodiments described herein.

FIG. 2A illustrates an exemplary garment according to embodiments described herein including a support band having a plurality of attachment points.

FIG. 2B illustrates the exemplary garment of FIG. 2A from a front perspective view.

FIG. 2C illustrates the exemplary garment of FIG. 2A with a support strap removably attached thereto.

### DETAILED DESCRIPTION

In the following description of preferred embodiments, reference is made to the accompanying drawings which form a part hereof, and in which it is shown by way of illustration specific embodiments in which the invention can be practiced. It is to be understood that other embodiments can be used and structural changes can be made without departing from the scope of the embodiments of this invention.

Exemplary embodiments described herein include a wearable device having a support band with a plurality of attachment locations. The wearable device may use non-restrictive, anatomically fit, elastomeric (stretchable) materials configured and attached together corresponding to achieve desired results. When worn, exemplary embodiments of a garment having features described herein may be used to interact with the shape and motion of the wearer to generate and/or deliver therapeutic modalities including, but not limited to, tactile biofeedback, muscle support, tensile resistance used to train, retrain, maintain, or otherwise improve the wearer's muscle balance, circulation, physical appeal, overall postural fitness, anatomical support, comfort, and combinations thereof. Exemplary embodiments may be used for muscle and postural training when worn intermittently.

Exemplary embodiments may be used with garments having a form fit, such as performance apparel, compression apparel, shapewear, maternal wear, among others. Exemplary embodiments may use, configure, and position materials and component panels to synergistically assist or correspond with contractual properties of the muscle groups and/or correspond or accommodate physical properties of the body feature in which they overlay, contact, or traverse. Exemplary embodiments may configure material sections to define or correspond to an anatomical form or correspond to respond to an anatomical attribute for which they overlay or for which they intent to influence.

Exemplary embodiments include any combination of the features disclosed herein. Exemplary embodiments may include a garment, such as a shirt or half top with the component parts integrated therein to create an integrated unit. Exemplary embodiments may include inserts or replacement panels to be incorporated into existing apparel and/or attached to existing apparel to provide some of the benefits described herein. Exemplary features described herein may be used to provide apparel that facilitates and corresponds to the physiological movement of the wearer. Exemplary embodiments may be used with other features known in the art, such as tensioning straps to provide selective tensions to the body. Exemplary embodiments may be used to avoid or reduce unnatural, or uncomfortable pressures to the wearer's body when in static and dynamic motion. Exemplary embodiments may be used for non-restrictive muscle support, neuromuscular activation, biofeedback, muscle training, comfort, support, and combinations thereof.

As used herein, the stretch of a material includes its ability to elongate in one or more directions upon application of a pulling or stretch force and return to approximately its original shape or length. The amount of stretch is determined by the amount the material stretches under a given force. Therefore, the greater the elongation for a given force, the greater the relative stretch. As used herein, the bias of stretch is the direction(s) in which a material may have a greater stretch than compared to other directions of stretch. For example, a material may have a single bias of stretch if it elongates to a greater extent under an application of force in a first direction, and elongates to a lesser extent under the same amount of force applied in any other direction.

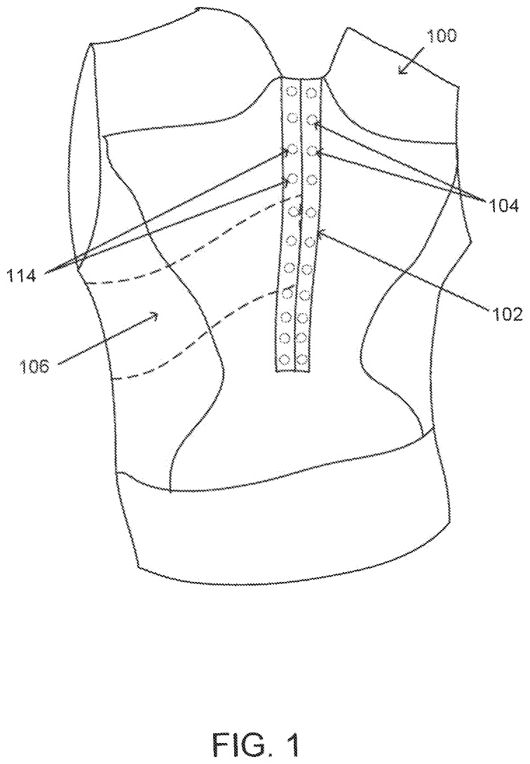

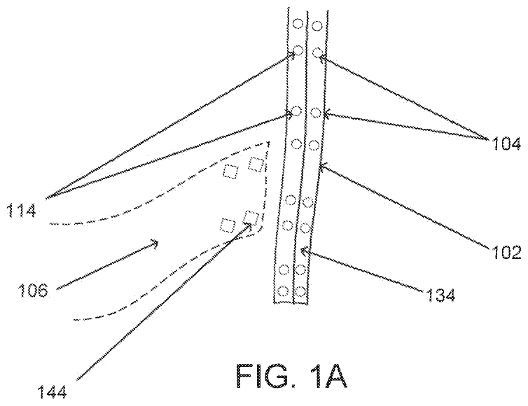

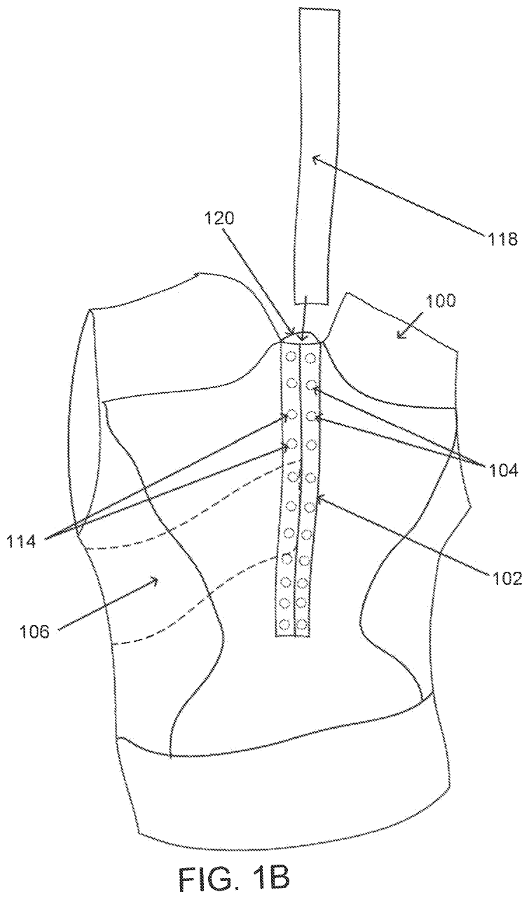

FIG. 1 illustrates an exemplary garment 100 having a support band 102 and a plurality of attachment points 104 thereon. The support band may be configured as a linear surface configured to support a series of attachment points 104. As shown, such as in FIG. 1B, the support band may extend a partial length of a garment, such as from a neck to a mid-back section of the wearer. The support band may also extend an entire length of the garment. In an exemplary embodiment, the support band having a plurality of attachment points may be positioned longitudinally along a length of the back side of a garment over a spine of a wearer when in a worn position. The support band may be used in any orientation or position in which removable attachment may be desired for the garment components or for customization and/or fitting.

The plurality of attachment points may be from any removable attachment. For example, the attachment point may be a button, mated surface, snap, magnetic attachment, hook/loop, or combinations thereof. As shown, the attachment points 104 is created between mated surface 114 in which a projection and indentation define a mated pair. The attachment point is also magnetic between the mated pair such that attachment is easily created and secured.

The plurality of attachment points may be positioned at a defined spacing between adjacent attachment points. The defined spacing may include skipped attachment points such that a series of adjacent attachment points can be easily identified and distinguished from adjacent sets of attachment points. In this way, the set of attachment points may be used to provide attachment instructions to a patient. For example, a patient may be informed to couple a strap as describe herein to first set of attachment points at points 2-3 or second set of attachment points at points 3-5. The mated surfaces or attachment point may also be coded to provide distinguishing identifiers. For example, the attachment points may be color coded or shape coded. One side of the mated surface may include circles, while the other side of mated surface may include squares. The instructions provided to a patient may therefore be to attach the circle interface to the square interface at positions 3-5 or to the green positions. Different combinations of indicators may be used to reposition the garment in a desired configuration, such as after removal or after washing to recreate a desired or customized experience for the user for repeated and repeatable use.

In an exemplary embodiment the garment includes one or more removable straps 106 for coupling to the support band at one or more of the attachment points. The strap may include an elastic band with one or more elastic bias of stretch. As shown, such as in FIG. 1A, the strap 106 may have a longitudinal bias of stretch in the length direction of the strap. The strap may include corresponding attachment points 144 for removable attachment to the attachment points 114 of the support band. An exemplary attachment configuration may be a-symmetric to impose an external pull on the user's spine.

In an exemplary embodiment, the straps 106 are configured to removably attach to the support band 102 over a spine of the wearer. The opposing end of the strap may be attached to a front side of the garment. The attachment at the front of the garment may be over or approximate to a rib cage of a wearer. The attachment of the strap at the front of the garment may be removable or permanent. By permanent, the attachment may still be removed, but is not considered easily reattachable. Therefore, permanent attachment may include sewing, bonding, adhering, or other attachment. The strap may be removably attached, such as through another support band positioned on the front of the garment. Other attachment mechanisms may also be used, such as through hook and loop, buckle, snap, button, mated surfaces, magnetic attraction, and combinations thereof. The strap is separate from the underlying garment and may be coupled only at the terminal ends or attachment points according to embodiments described herein. The separation between the strap and the garment may permit dynamic and relative movement of the strap relative to the garment and wearer.

In an exemplary embodiment, a strap may be positioned between any combination of attachment points. For example, a strap may traverse the spinal support band and instead coupled between attachment points on the front of the garment and extend around the user on a back side thereof. Exemplary embodiments may also include selectable attachment across any combination of attachments points such that a strap may have different attachment points along its length to attach to different locations on the garment and/or at one, two, three, or more attachment locations on different regions of the wearer's body and each region may include one, two, three, or any combination of attachment points within the proximity of the region.

An exemplary embodiment of a garment may include a pocket configured to removably receive a strap. The pocket may be configured to be positioned between one or more different attachment points according to embodiments described herein to assist in the relative positioning of a strap on a wearer's body during an in use configuration. The pocket may also support a strap that may be attached to itself, the garment, other straps, or other features objects. As shown, a pocket is positioned along a backside of the garment traversing across the garment between lateral sides of the garment such that the pocket traverses a lower back or lower back pelvic area of a wearer.

As illustrated in FIG. 1B, in an exemplary embodiment, a garment 100 may include a pocket 120 positioned between or along a support band 102 such that a plurality of attachment points 104 are positioned over the pocket 120. The pocket may be configured to removably receive an insert 118 of desired rigidity having a defined shape. The pocket may therefore support an insert to be used as a splint or shaped object in which the body is intended to conform or partially conform or in which the body is intended to be supported thereby. The pocket may extend along an entire or partial length of the garment. The pocket may be along any portion of the garment and may be with or without a support band and/or a plurality of attachment points. In an exemplary embodiment, the pocket is configured to extend and be positioned over a portion of the wearer's spine. In an exemplary embodiment, the insert and/or interior surface of the packet may include attachment features to retain the insert in a desired relative position to the garment and/or pocket during use. The attachment feature may include tethered attachment, hook and loop, mated surfaces, magnetic attraction, snap, buttons, and combinations thereof.

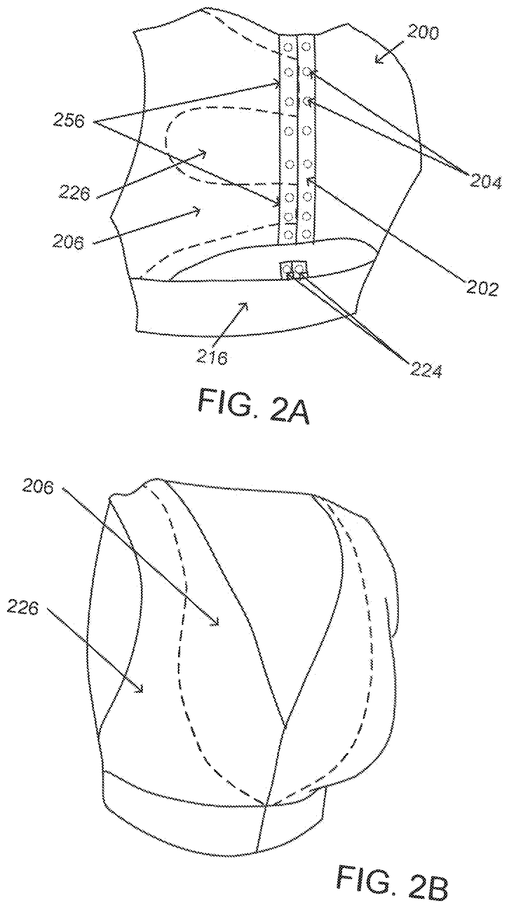

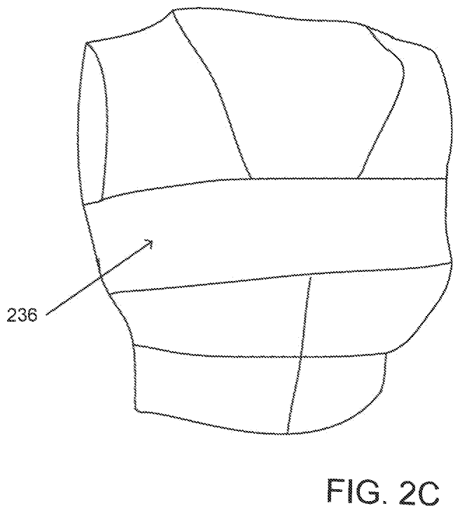

FIG. 2A illustrates an exemplary embodiment of a garment 200 similar to FIG. 1 having a support band 202. The support band provides for a plurality of attachment points 204 for coupling a strap 206. The strap may have different configurations, and is not limited to a linear, elongated configuration. As seen in FIG. 2A, the strap 206 may include a plurality of attachment ends emanating from the same strap. As shown, each strap is couple to the support band 202 at two different ends, each end having a plurality of attachment points 256. The attachment ends of the strap are separated 226 from each other, such that the strap is configured to attach to the support band at opposing end regions of the support band 202. The other end of the strap is configured to attach to the front of the garment.

As illustrated in FIG. 2A, exemplary embodiments may also include a configurable garment section 216 that is configured to circumscribe or partially circumscribe a wearer and couple to one or more of the support bands 202 described herein. The configurable garment section 216 may include one or more attachment points 224 on the garment that is configured to coupled to one or more of the attachment points 204 of a support band 202 to customize a size or configuration of the garment. The attachment of one attachment point to another attachment point may therefore be used to take up additional material slack or impose a greater elastic pull on the garment when in a use configuration.

FIGS. 2A-2B illustrate an exemplary multiple layer garment according to embodiments described herein. As shown, a first layer 226 may provide a support layer for the garment. The support layer may couple or integrally support one or more support bands 202 having one or more attachment points 204 thereon. The second layer 206 may integrate one or more straps for configurable attachment to the support bands of the first layer. As shown, an inner layer may be configured to providing support and/or shaping of a wearer's body parts. As seen in FIG. 2B, for a female application, the inner layer 226 may provide lateral, outer side support and/or under cup support for a wearer's breast and extend over the shoulder of the wearer and around a lateral side of the wearer to the back of the garment and support the support band with a plurality of attachment points thereon. An outer layer 206 may provide a front coverage that covers the wearer's body parts, such as the breast of the wearer and may support on a back side of the wearer, the one or more straps for removable attachment to the support band.

Exemplary embodiments of the garments provided herein may include a zipper attachment at the front or other location for removable placement of the garment on the wearer. Other configurations of opening or separation of the garment for a wearer may also be used, such as through buttons, hook and loop, snaps, mated surfaces, magnetic attraction, support band having a plurality of attachment points, and combinations thereof. The separation of the garment may permit easier adornment of the garment on the wearer while the garment is in a preconfigured setting. In this way, the user and/or practioner may set the customization of the garment to impose desired pulls on the wearer in the use configuration, while still permitting the wearer to put the garment on and take the garment off without interfering with the desired settings.

Exemplary embodiments described herein may be used with additional straps. As shown in FIG. 2C, strap 236 may be positioned above and/or below the wearer's breast to impose an external compressive or desired shaping force on the breast of the wearer. The strap(s) 236 may be elongated straps having opposing terminal ends, each end positioned and/or attached at one or more attachment points on one or more support bands. As shown, the straps may extend around the wearer and having terminal ends attached at the same support band or at a support band positioned over a wearer's spine in a use configuration. Such configurations may be used pre and post-surgery or other medical procedure to assist in healing and shaping of a wearer.

Exemplary embodiments may be used with interchangeable straps and/or with a single strap. For example, a single strap may include a plurality of attachment points such that the same strap may be positioned in a desired location and impose a desired stretch on the wearer in a worn configuration by selecting a combination of attachment points from the strap and/or the garment. Multiple straps may also be used interchangeably with the same garment to provide the desired customization. Accordingly, straps of various size and/or stretch factors may be provided and used interchangeably with a garment to create the desired effect and/or customization for a wearer.

Exemplary embodiments of a support layer described herein may be used alone or in conjunction with another layer, such as one, two, or more outer panels described herein. The support layer may be configured and positioned to provide support and/or shaping to the soft tissue of the breast of the wearer. As shown, the support layer is configured to attach along a bottom edge to the lower band. The support layer is configured to extend around an outer edge of the breast at may attach along a lateral side of the garment and extend up and over the shoulder of the wearer. The support layer may attach on a back side of the wearer.

As illustrated, two outer panels may be configured to extend over a top of the shoulder of the wearer and attach to each other and/or to a band coupled to the lower band of the front portion. The two outer panels may couple directly or indirectly through one or more extension bands to the lower band. The two outer panels may be directly attached to each other at the seam to the lower band or extension band and/or may be directly coupled in a position away from the seam to another band.

Exemplary embodiments of a band and/or strap described herein may include woven or knit material infused with an elastic. In an exemplary embodiment, a method of making a band may include providing a fabric material such as a knit or woven material and a sheet of an elastic material. In an exemplary embodiment, the fabric and the sheet may be overlayed over each other. In an exemplary embodiment, the bias of stretch of the sheet and the fabric may be positioned to desired orientation. The bias of stretch of the sheet and the fabric may be parallel, oblique, perpendicular, or other orientation. In an exemplary embodiment, the elastic material is heated to infuse the elastic in the fabric. In an exemplary embodiment, the fabric may comprise nylon or a nylon blend. The elastic may be an elastomer, such as spandex.

In an exemplary embodiment, methods, components, and features of the embodiment may be used and/or integrated into features of the garments as described herein. For example, the methods of creating a band may be used to replace seams of a garment and/or may be used as a stabilizer for portions of the garment. In an exemplary embodiment, garments described herein may incorporate stabilizers at peripheral edges of the garment. The stabilizer may be created in methods and ways similar to the band described herein. The stabilizer may be incorporated around the peripheral edges of the support layer. Exemplary embodiments of the stabilizer may be incorporated in a garment at an arm aperture, neck aperture, waist aperture, and combinations thereof. In an exemplary embodiment, the method of creating a band may be used to attach one or more panels together. Therefore, instead of providing a single layer or single fabric material, the method may include providing two portions or edges of a fabric, such as from the same or different panels. The first and second fabric portions may thereafter be overlayed with respect to each other and the elastic sheet. The elastic sheet may be positioned between the fabric layers or over the fabric layers. The elastic sheet may then be heated to infuse the elastic into the first and second fabric layers.

The connection between the garment and portions thereof may be through snap, button, hood and loop, magnetic, mated surfaces, frictional engagement, tying, sewing, bonding, adhesion, or other method, and combinations thereof.

Exemplary embodiments described herein are exemplary only. Features from any garment may be used alone or in any combination to create a garment within the scope of the instant disclosure. Exemplary features that may be used alone or in combination as described herein may include any combination of the follow or any feature described herein.

Exemplary embodiments of garments described herein provide for the customization of a garment for an individual wearer using the same basic garment. For example, a garment for use on an upper body may have a given size for use with different wearer's spanning a size and/or configuration range and/or with different attributes to be influenced by the garment. The garment may include a plurality of attachment points through one or more support bands for the removable attachment of one or more straps therebetween and/or the removable attachment of different portions of the garment.

Exemplary embodiments described herein incorporate non-linear apparel construction. For example, embodiments described herein may be created by patterns and/or panels with linear portions or edges. Embodiments described herein may include patterns and/or panels without any linear portions or edges. Embodiments described herein may include patterns and/or panels in which edges configured to attach to another portion or panel consists of only curved, non-linear edges. In an exemplary embodiment, panels may be attached to avoid or minimize perpendicular seams, perpendicular panels, or perpendicular patterned pieces.

Exemplary embodiments described herein may comprise materials having a systematic property of elasticity including single and/or multi-directional orientations. In an exemplary embodiment, materials are knit or woven in an oblique orientation to avoid or reduce perpendicular alignment of the fibers. Exemplary embodiments comprise a warp weave with or without a weft.

Exemplary embodiments orient material panels such that a direction of a bias of stretch are oriented with respect to and/or aligned with a portion of the underlying muscle group for which the material is intended to overlay when in a worn position on a wearer. Exemplary embodiments include configurations, orientations, and material configured to mimic the natural underlying physiologic properties of the neuromuscular movement of the wearer. Exemplary embodiments include panels, orientations, configurations, and materials to create anatomic anchoring points such that portions of the garment correspond to underlying anatomic areas to provide a natural anchor of the garment relative to the body of the user when in a worn position.

In an exemplary embodiment, a garment may include a front panel with greater stretch to permit the expansion or variation of the breast areas of a user. In an exemplary embodiment, the bias of stretch of the front portion of the garment configured to overlay the soft tissues of the breast section of the wearer is in an oblique or diagonal direction relative to the vertical alignment of the body direction. In an exemplary embodiment, the bias of stretch may be configured such that the garment is configured to stretch approximately or at least 25% more in the direction of the bias of stretch than in other directions. Other bias of stretch described herein may include a similar or the same 25% bias of stretch factor.

In an exemplary embodiment, a garment may include a zipper portion. The zipper portion may extend from a top or neck aperture of the garment downward toward a lower edges of the garment. The zipper may extend the full or only a partial distance along the length of the garment.

Exemplary embodiments may comprise garments configured to manipulate the outer muscles of a wearer in order to influence the inner muscles groups of the wearer. Exemplary embodiments may be used by a wearer directly in contact with the skin of the wearer and/or over other layers of a wearer such as over other clothing or garments of a wearer.

Exemplary embodiment of a garment for use with a female body may include additional anchor panels or extensions on anchor panels to support the added frontal weight of the female body. For example, an anchor panel may circumscribe the body at approximately the middle to lower portion of the rib cage when the garment is in a worn position. The anchor panel may extend around and across the front of the garment. The anchor panel may extend across and thereby be disconnect by the zipper or may be unobstructed by a zipper. The anchor panel may extend around the back of the wearer and at or on an area between the shoulder blades of the wearer when the garment is in a worn position. The anchor panel may comprise a band circumscribe the wearer below the breast of the wearer.

In an exemplary embodiment, garments according to embodiments described herein may include multiple layers. Layers of the garment may be used to stabilize, shape, impose greater effects on the wearer and combinations thereof. For example, a shaping layer may be used to move a portion of the wearer's body such as a breast to a desired location when the garment is in a worn position. For example, a support layer may be used such as across a back of a garment. The support layer may include an aperture and attachment points to the garment. Layers may also be added to provide coverage to anatomic areas of the body, such as over private areas such as breasts.

In an exemplary embodiment of a garment, a front area of the garment may include material and/or panel of greater stretch, while a back portion includes an area of less stretch. The area of less stretch may define an anchor zone. The front area and back area may be on a waist section of the garment. In an exemplary embodiment, extensions from the front area and back area may overlap at lateral sides to define lateral portions of the garment.

Exemplary embodiments comprise woven or knit materials infused with an elastic material. Woven or knit materials may include nylon. Elastic materials may include lycra, spandex, elastomer, etc. Exemplary embodiments comprise materials having a warp weave and/or warp knit with and without a weft. In an exemplary embodiment, a weft may comprise titanium strands.

In an exemplary embodiment, one or more and/or all of the seams of a garment may be bonded. In an exemplary embodiment, one or more and/or all of the seams of the garment may not be sewn. In an exemplary embodiment seams may be bonded by integrating a material into and between the panels of the seam.

What is claimed is:

1. A garment, comprising:
a front portion;
a back portion; and
a support positioned on the back portion, the support having a plurality of attachment points thereon.

2. The garment of claim 1, further comprising:
a strap permanently attached to the front portion, the back portion, or both the front and back portion, the strap having a plurality of attachment points.

3. The garment of claim 2, wherein the plurality of attachment points on the support and the plurality of attachment points on the strap comprise mated surfaces.

4. The garment of claim 3, wherein any one of the plurality of attachment points on the strap is configured to mate with any one of the plurality of attachment points on the support.

5. The garment of claim 4, wherein the mated surface comprise mated indentations and projections.

6. The garment of claim 5, wherein the plurality of attachment points of the strap and the plurality of attachment points of the support are magnetic.

7. The garment of claim 1, further comprising a strap circumferentially positioned around a bottom portion of the garment and coupled to the front position and the back portion.

8. The garment of claim 7, wherein a gap is provided between the strap and the support.

9. The garment of claim 8, wherein the strap comprises an attachment point configured to removably couple to the attachment points of the support.
